(19) United States
(12) Patent Application Publication
(10) Pub. No.: US 2025/0319312 A1
(43) Pub. Date: Oct. 16, 2025

(54) PATHWAYS TO PAIN RELIEF VIA ADAPTIVE ELECTRICAL NEUROSTIMULATION TREATMENT

(71) Applicant: Boston Scientific Neuromodulation Corporation, Valencia, CA (US)

(72) Inventors: Carla Agurto, Ossining, NY (US); Erhan Bilal, Westport, CT (US); Pritish R. Parida, Cortland Manor, NY (US); Jenna Reinen, Rye Brook, NY (US); Jeffrey Rogers, Briarcliff Manor, NY (US); Tigran Tchrakian, Castleknock (IE)

(21) Appl. No.: 19/098,565

(22) Filed: Apr. 2, 2025

Related U.S. Application Data
(60) Provisional application No. 63/633,954, filed on Apr. 15, 2024.

(51) Int. Cl. A61N 1/36; A61N 1/372; G16H 20/40; G16H 50/70

(52) U.S. Cl. CPC A61N 1/36071; A61N 1/36062; A61N 1/36078; A61N 1/36132; A61N 1/36178; A61N 1/37247; A61N 1/37264; G16H 20/40; G16H 50/70

(57) ABSTRACT

Systems and techniques to determine programming of an implantable electrical neurostimulation device, through chronic pain treatment modeling that evaluates pain experience states and transitions for a patient. In an example, a system to determine programming of a neurostimulation device performs operations to: determine possible pathways to traverse pain experience states of a chronic pain condition, as the possible pathways provide respective paths among the states from a starting state to one or more intermediate states to a goal state; determine transition costs between the states used in the possible pathways, with respective pain experience states being associated with different pain management characteristics of neurostimulation therapy; identify a path of the possible pathways to reach the goal state, based on the transition costs and patient characteristics; and select programming parameters for the neurostimulation device, to provide neurostimulation therapy based on the identified path to achieve the goal state.

### CLAIM OF PRIORITY

This application claims the benefit of U.S. Provisional Application No. 63/633,944, filed on Apr. 15, 2024, which is hereby incorporated by reference in its entirety.

### DISCLAIMER

The claims and scope of the subject application, and any continuation, divisional or continuation-in-part applications claiming priority to the subject application, are solely limited to embodiments (e.g., systems, apparatus, methodologies, computer program products and computer readable storage media) directed to implanted electrical stimulation for pain treatment and/or management.

### STATEMENT REGARDING JOINT RESEARCH AND DEVELOPMENT

The present subject matter was developed and the claimed invention was made by or on behalf of Boston Scientific Neuromodulation Corporation and International Business Machines Corporation, parties to a joint research agreement that was in effect on or before the effective filing date of the claimed invention, and the claimed invention was made as a result of activities undertaken within the scope of the joint research agreement.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable electrical neurostimulator, also referred to as an implantable pulse generator (IPG), and one or more implantable leads each including one or more electrodes. The implantable electrical neurostimulator delivers neurostimulation energy through one or more electrodes placed on or near a target site in the nervous system.

A neuromodulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to create an analgesic effect that masks pain sensation. While modern electronics can accommodate the need for generating and delivering stimulation energy in a variety of forms, the capability of a neurostimulation system depends on its post-manufacturing programmability to a great extent. For example, a sophisticated neurostimulation program may only benefit a patient when it is customized for that patient, and when it provides sufficient pain benefits that are balanced against other quality of life considerations for the patient such as sleep quality, mobility, mood, and the like. This is further complicated because specific programs and programming settings may lead to different outcomes on these quality of life considerations for a specific patient when applied at different times—even as the benefit of particular programming settings and types of treatments changes over time.

### SUMMARY

This Summary includes examples that provide an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

Example 1 is a system for determining programming of an implantable electrical neurostimulation device of a human patient for treating a chronic pain condition, the system comprising: a processor; and a memory device comprising instructions, which when executed by the processor, cause the processor to perform operations that: determine possible pathways to traverse pain experience states of the chronic pain condition, wherein the possible pathways provide respective paths among the pain experience states from a starting state to one or more intermediate states to a goal state; determine transition costs between the pain experience states involved in each of the possible pathways, wherein respective states of the pain experience states are associated with different pain management characteristics based on therapy with the neurostimulation device; identify a path of the possible pathways to reach the goal state, based on the transition costs and characteristics of the human patient; and select programming parameters for use in the neurostimulation device of the human patient to cause a neurostimulation therapy, based on the identified path to achieve the goal state.

In Example 2, the subject matter of Example 1 optionally includes subject matter where the pain experience states are determined based on pain experience data collected from a population of patients.

In Example 3, the subject matter of Example 2 optionally includes subject matter where the transition costs are determined based on a frequency of transitions associated with the population of patients.

In Example 4, the subject matter of any one or more of Examples 1-3 optionally include subject matter where to determine the possible pathways includes to identify the pain experience states and relative rankings of the pain experience states, and wherein the relative rankings are customized to the human patient.

In Example 5, the subject matter of Example 4 optionally includes subject matter where the relative rankings are customized to the human patient based on a measurement of: one or more preference associated with the human patient, or a resilience of the human patient.

In Example 6, the subject matter of any one or more of Examples 1-5 optionally include subject matter where to identify the path to reach the goal state for the human patient includes use of a path traversal strategy provided from one of: a shortest path independent of the transition costs from the starting state to the goal state; a path with a lowest total cost of the transition costs to traverse from the starting state to the goal state; a path with a lowest initial cost of the transition costs to traverse from the starting state to a first of the one or more intermediate states; or a path with a lowest individual transition costs to traverse from the starting state to the one or more intermediate states to the goal state.

In Example 7, the subject matter of Example 6 optionally includes subject matter where the path traversal strategy is selected for the human patient based on one or more preference associated with the human patient or resilience associated with the human patient.

In Example 8, the subject matter of any one or more of Examples 1-7 optionally include subject matter where the pain experience states are associated with defined attributes based on one or more of: medication management, pain level, emotional state, or mobility.

In Example 9, the subject matter of any one or more of Examples 1-8 optionally include subject matter where the goal state is associated with an attribute that provides an improvement of one or more of: a sleep state, a mobility state, a medication state, an emotional state, or a pain level measurement.

In Example 10, the subject matter of any one or more of Examples 1-9 optionally include subject matter where to select the programming parameters for use in the neurostimulation device of the human patient includes a selection or recommendation of one or more program that includes the programming parameters; and wherein each of the respective states used in the possible pathways is associated with a separate program used for the neurostimulation device that includes respective combinations of the programming parameters.

In Example 11, the subject matter of any one or more of Examples 1-10 optionally include subject matter where the instructions further cause the processor to perform operations that: output the programming parameters to the neurostimulation device of the human patient, wherein deployment of the programming parameters within one or more neurostimulation program causes a change in operation of the implantable electrical neurostimulation device.

In Example 12, the subject matter of any one or more of Examples 1-11 optionally include subject matter where the programming parameters cause a change in programming for the implantable electrical neurostimulation device for one or more of: pulse patterns, pulse shapes, a spatial location of pulses, waveform shapes, or a spatial location of waveform shapes, for modulated energy provided with a plurality of leads of the implantable electrical neurostimulation device.

In Example 13, the subject matter of any one or more of Examples 1-12 optionally include subject matter where the implantable electrical neurostimulation device is further configured to treat the chronic pain condition by delivering at least one of: an electrical spinal cord stimulation, an electrical brain stimulation, or an electrical peripheral nerve stimulation, in the human patient.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a method, performed by a computing device to determine programming of an implantable electrical neurostimulation device for treating a chronic pain condition of a human patient, the method comprising: determining possible pathways to traverse pain experience states of the chronic pain condition, wherein the possible pathways provide respective paths among the pain experience states from a starting state to one or more intermediate states to a goal state; determining transition costs between the pain experience states involved in each of the possible pathways, wherein respective states of the pain experience states are associated with different pain management characteristics based on therapy with the neurostimulation device; identifying a path of the possible pathways to reach the goal state, based on the transition costs and characteristics of the human patient; and selecting programming parameters for use in the neurostimulation device of the human patient to cause a neurostimulation therapy, based on the identified path to achieve the goal state.

In Example 17, the subject matter of Example 16 optionally includes subject matter where the pain experience states are determined based on pain experience data collected from a population of patients, and wherein the transition costs are determined based on a frequency of transitions associated with the population of patients.

In Example 18, the subject matter of any one or more of Examples 16-17 optionally include subject matter where determining the possible pathways includes identifying the pain experience states and relative rankings of the pain experience states, and wherein the relative rankings are customized to the human patient.

In Example 19, the subject matter of Example 18 optionally includes subject matter where the relative rankings are customized to the human patient based on a measurement of: one or more preference associated with the human patient, or a resilience of the human patient.

In Example 20, the subject matter of any one or more of Examples 16-19 optionally include subject matter where identifying the path to reach the goal state for the human patient includes use of a path traversal strategy provided from one of: a shortest path independent of the transition costs from the starting state to the goal state; a path with a lowest total cost of the transition costs to traverse from the starting state to the goal state; a path with a lowest initial cost of the transition costs to traverse from the starting state to a first of the one or more intermediate states; or a path with a lowest individual transition costs to traverse from the starting state to the one or more intermediate states to the goal state.

In Example 21, the subject matter of Example 20 optionally includes subject matter where the path traversal strategy is selected for the human patient based on one or more preference associated with the human patient or resilience associated with the human patient.

In Example 22, the subject matter of any one or more of Examples 16-21 optionally include subject matter where the pain experience states are associated with defined attributes based on one or more of: medication management, pain level, emotional state, or mobility; and wherein the goal state is associated with an attribute that provides an improvement of one or more of: a sleep state, a mobility state, a medication state, an emotional state, or a pain level measurement.

In Example 23, the subject matter of any one or more of Examples 16-22 optionally include subject matter where selecting the programming parameters for use in the neurostimulation device of the human patient includes a selection or recommendation of one or more program that includes the programming parameters; and wherein each of the respective states used in the possible pathways is associated with a separate program used for the neurostimulation device that includes respective combinations of the programming parameters.

In Example 24, the subject matter of any one or more of Examples 16-23 optionally include outputting the programming parameters to the neurostimulation device of the human patient, and wherein deployment of the programming parameters within one or more neurostimulation program causes a change in operation of the implantable electrical neurostimulation device.

In Example 25, the subject matter of any one or more of Examples 16-24 optionally include subject matter where the implantable electrical neurostimulation device is further configured to treat the chronic pain condition by delivering at least one of: an electrical spinal cord stimulation, an electrical brain stimulation, or an electrical peripheral nerve stimulation, in the human patient; and wherein the programming parameters cause a change in programming for the implantable electrical neurostimulation device for one or more of: pulse patterns, pulse shapes, a spatial location of pulses, waveform shapes, or a spatial location of waveform shapes, for modulated energy provided with a plurality of leads of the implantable electrical neurostimulation device.

Example 26 is a device to determine programming of an implantable electrical neurostimulation device for treating a chronic pain condition of a human patient, the device comprising: at least one processor and at least one memory; pain experience state evaluation circuitry, operable with the at least one processor and the at least one memory, configured to: determine possible pathways to traverse pain experience states of the chronic pain condition, wherein the possible pathways provide respective paths among the pain experience states from a starting state to one or more intermediate states to a goal state; determine transition costs between the pain experience states involved in each of the possible pathways, wherein respective states of the pain experience states are associated with different pain management characteristics based on therapy with the neurostimulation device; identify a path of the possible pathways to reach the goal state, based on the transition costs and characteristics of the human patient; and neurostimulation programming circuitry, in operation with the at least one processor and the at least one memory, configured to: select programming parameters for use in the neurostimulation device of the human patient to cause a neurostimulation therapy, based on the identified path to achieve the goal state.

In Example 27, the subject matter of Example 26 optionally includes subject matter where the pain experience states are determined based on pain experience data collected from a population of patients, and wherein the transition costs are determined based on a frequency of transitions associated with the population of patients.

In Example 28, the subject matter of any one or more of Examples 26-27 optionally include subject matter where to determine the possible pathways includes to identify the pain experience states and relative rankings of the pain experience states, and wherein the relative rankings are customized to the human patient.

In Example 29, the subject matter of Example 28 optionally includes subject matter where the relative rankings are customized to the human patient based on a measurement of: one or more preference associated with the human patient, or a resilience of the human patient.

In Example 30, the subject matter of any one or more of Examples 26-29 optionally include subject matter where to identify the path to reach the goal state for the human patient includes use of a path traversal strategy provided from one of: a shortest path independent of the transition costs from the starting state to the goal state; a path with a lowest total cost of the transition costs to traverse from the starting state to the goal state; a path with a lowest initial cost of the transition costs to traverse from the starting state to a first of the one or more intermediate states; or a path with a lowest individual transition costs to traverse from the starting state to the one or more intermediate states to the goal state.

In Example 31, the subject matter of Example 30 optionally includes subject matter where the path traversal strategy is selected for the human patient based on one or more preference associated with the human patient or resilience associated with the human patient.

In Example 32, the subject matter of any one or more of Examples 26-31 optionally include subject matter where the pain experience states are associated with defined attributes based on one or more of: medication management, pain level, emotional state, or mobility; and wherein the goal state is associated with an attribute that provides an improvement of one or more of: a sleep state, a mobility state, a medication state, an emotional state, or a pain level measurement.

In Example 33, the subject matter of any one or more of Examples 26-32 optionally include subject matter where to select the programming parameters for use in the neurostimulation device of the human patient includes a selection or recommendation of one or more program that includes the programming parameters; and wherein each of the respective states used in the possible pathways is associated with a separate program used for the neurostimulation device that includes respective combinations of the programming parameters.

In Example 34, the subject matter of any one or more of Examples 26-33 optionally include subject matter where the neurostimulation programming circuitry is further configured to output the programming parameters to the neurostimulation device of the human patient, and wherein deployment of the programming parameters within one or more neurostimulation program causes a change in operation of the implantable electrical neurostimulation device.

In Example 35, the subject matter of any one or more of Examples 26-34 optionally include subject matter where the implantable electrical neurostimulation device is further configured to treat the chronic pain condition by delivering at least one of: an electrical spinal cord stimulation, an electrical brain stimulation, or an electrical peripheral nerve stimulation, in the human patient; and wherein the programming parameters cause a change in programming for the implantable electrical neurostimulation device for one or more of: pulse patterns, pulse shapes, a spatial location of pulses, waveform shapes, or a spatial location of waveform shapes, for modulated energy provided with a plurality of leads of the implantable electrical neurostimulation device.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a neurostimulation system.

FIG. 2 illustrates, by way of example, an embodiment of a stimulation device and a lead system, such as may be implemented in the neurostimulation system of FIG. 1.

FIG. 3 illustrates, by way of example, an embodiment of a programming device, such as may be implemented in the neurostimulation system of FIG. 1.

FIG. 4 illustrates, by way of example, an implantable neurostimulation system and portions of an environment in which the system may be used.

FIG. 5 illustrates, by way of example, an embodiment of an implantable stimulator and one or more leads of a neurostimulation system, such as the implantable neurostimulation system of FIG. 4.

FIG. 6 illustrates, by way of example, an embodiment of a patient programming device for a neurostimulation system, such as the implantable neurostimulation system of FIG. 4.

FIG. 7 illustrates, by way of example, sequential operations for evaluating pain experience states in connection with neurostimulation therapy.

FIG. 8 illustrates, by way of example, a block diagram of a pain treatment modeling system used for evaluating and recommending programming parameters of a neurostimulation therapy based on pain experience states.

FIG. 9 illustrates, by way of example, pain experience states, pain experience state transitions, and pain improvement pathways evaluated in connection with a neurostimulation therapy.

FIG. 10 illustrates, by way of example, a mapping of pain state transitions and pathways in connection with a neurostimulation therapy.

FIG. 11 illustrates, by way of example, a matrix of data values from multiple users in connection with the evaluation of pain state transitions.

FIG. 12 illustrates, by way of example, a block diagram of a system for evaluating and recommending programming parameters of a neurostimulation therapy based on pain experience states.

FIG. 13 illustrates, by way of example, an embodiment of a processing method implemented by a system or device to evaluate pain experience states, in connection with programming of an implantable electrical neurostimulation device for treating chronic pain of a human subject.

FIG. 14 illustrates, by way of example, a block diagram of an embodiment of a computing system implementing pain state determination circuitry for use to evaluate programming of an implantable electrical neurostimulation device for treating chronic pain of a human subject.

FIG. 15 illustrates, by way of example, a block diagram of an embodiment of a computing system implementing neurostimulation programming circuitry for use to implement programming of an implantable electrical neurostimulation device for treating chronic pain of a human subject.

FIG. 16 is a block diagram illustrating a machine in the example form of a computer system, within which a set or sequence of instructions may be executed to cause the machine to perform any one of the methodologies discussed herein, according to an example embodiment.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present disclosure relates generally to medical devices, and more particularly, to systems, devices, and methods for electrical stimulation programming techniques, to perform implanted electrical stimulation for pain treatment and/or management. Specifically, this document discusses various techniques that can be used to identify, evaluate, and generate programming values of an implantable electrical neurostimulation device, for the treatment of chronic pain of a human subject (e.g., a patient). As an example, various systems and methods are described to identify a sequence of neurostimulation therapy states that a human subject should traverse, in order to achieve the best possible relief of chronic pain symptoms or pain experience. This sequence of neurostimulation therapy states may include different levels, types, and locations of stimulation treatment so that the patient's pain experience can be incrementally yet successfully improved. In determining the “best possible” outcome for a chronic pain condition, related aspects of a patient state and quality of life are considered in addition to a measurement of pain, such as mobility, sleep quality, medication management, mood or emotional state, and the like.

Chronic pain is a common condition for many patients, but which may be addressed through the use of neurostimulation therapy (e.g., electrical spinal cord stimulation, electrical peripheral nerve stimulation, or electrical brain stimulation) to deliver treatment. One limiting factor for existing applications of neurostimulation therapies is that, even if a number of advanced programs can be applied by a neurostimulation device, patients often only end up using a small number of the available treatments (e.g., only a small number of programs), and may not progress to the best outcome available from neurostimulation. Chronic pain improvement is complicated because pain treatment sometimes requires using different programs at different times to address different types or levels of symptoms, all while trying to progress the patient to reach an improved pain experience state. This is further complicated because the pain condition and pain perception in each patient is unique, and different patients do not necessarily follow the same path of pain improvement. However, individual patients do share certain conditions, behaviors, or attributes with others in a population of patients, so outcomes and experiences from a larger population can be used to direct what therapy to use and when to use this therapy.

The present inventive techniques and systems improve pain treatment scenarios through the use of a pain treatment modeling built around the patient, adapted to the state of the patient and state pathways determined from a patient population. This pain treatment modeling system is able to identify and implement new programs and program settings for the patient to apply for his or her specific conditions, predicted conditions, and relevant constraints and uncertainties of the operation of the neurostimulation device. This provides a benefit over current approaches for chronic pain treatment and neurostimulation programming that are based on human judgment or trial-and-error, or rigid schedules. Many types of prior chronic pain treatment approaches are focused on reducing self-reported pain as quickly as possible. However, not all patients reach a minimal pain level after treatment, and chronic pain has a strong psychological aspect that is mostly ignored by current practices.

Effective treatment of chronic pain with neurostimulation involves consideration of states and state transitions that are built up over time. The pain treatment modeling system discussed herein enables the analysis of multiple aspects of relevant treatment inputs, stages, and transitions, to enable a patient, caregiver, or medical professional to select, evaluate, modify, and implement certain programming parameters (e.g., settings) for a neurostimulator at different therapy stages or states. These programming parameters may be arranged or defined (e.g., created, modified, activated, etc.) into new or updated sets of neurostimulation operational programs (also plainly referred to as “programs” in this document), resulting use of a particular neurostimulation program that includes at least a portion of the pain treatment parameters identified as a best-fit for the human patient at a particular stage in a treatment pathway.

The techniques of this document can enable a human subject (e.g., patient) or a party/entity on behalf of the human subject to create, establish, activate, select, modify, update, or adapt a program for a device (or to re-program a device) within an expanded set of programs and program settings, to improve chronic pain treatment and treatment efficacy of neurostimulation device uses. In the approaches discussed below, machine learning techniques are used to identify common conditions experienced in a population data set, to determine similarities (and costs) in the transitions between treatment of pain states. In some examples, these pain states are defined by clustering longitudinal data collected from subjects along at least the following dimensions: pain, physical activity, sleep, medications, mood, and alertness. Other types of patient state or experience data may also be considered. Given the large number of permutations in neurostimulation output available in any given program—and the wide variation among different types of programs—the consideration of a particular patient's pain experience state and pain therapy pathway is not feasible with existing approaches.

By way of example, operational parameters of the electrical neurostimulation device may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The electrical neurostimulator may use current or voltage sources to provide the neurostimulator output, and apply any number of control techniques to modify the electrical stimulation applied to anatomical sites or systems related to chronic pain. In various embodiments, a neurostimulator program may include parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

In various embodiments, the present subject matter may be implemented using a combination of hardware and software designed to provide users such as patients, caregivers, clinicians, researchers, physicians, or others with the ability to generate, identify, select, implement, and update neurostimulation programs based on chronic pain experience states and state transitions. The adaptation of neurostimulation programs used for different states may provide variation in the location, intensity, and type of defined waveforms and patterns in an effort to increase therapeutic efficacy and/or patient satisfaction for therapies, including but not limited to SCS and DBS therapies. While neurostimulation is specifically discussed as an example, the present subject matter may apply to similar therapy that employs stimulation or modulated pulses of electrical or other forms of energy for treating chronic pain.

The delivery of neurostimulation energy that is discussed herein may be delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, neural signals may include more sophisticated patterns to communicate various types of information, including sensations of pain, pressure, temperature, etc. Accordingly, the following drawings provide an introduction to the features of an example neurostimulation system and how such programming may be accomplished through neurostimulation systems.

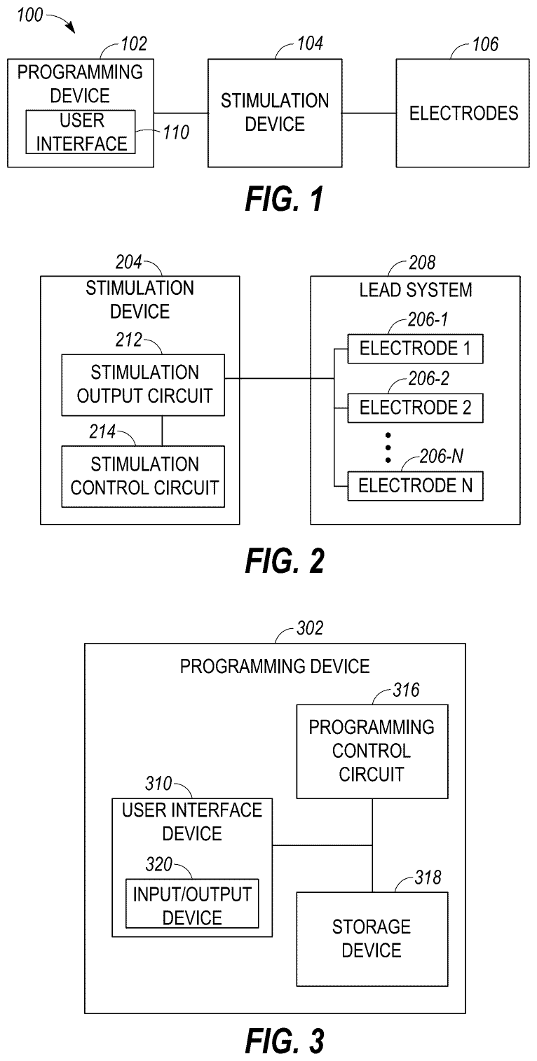

FIG. 1 illustrates an embodiment of an electrical neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets through electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a clinical user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In various embodiments, programming device 102 includes a user interface 110 (e.g., a user interface embodied by a graphical, text, voice, or hardware-based user interface) that allows the user to set and/or adjust values of the user-programmable parameters by creating, editing, loading, and removing programs that include parameter combinations such as patterns and waveforms. These adjustments may also include changing and editing values for the user-programmable parameters or sets of the user-programmable parameters individually (including values set in response to a therapy efficacy indication). Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

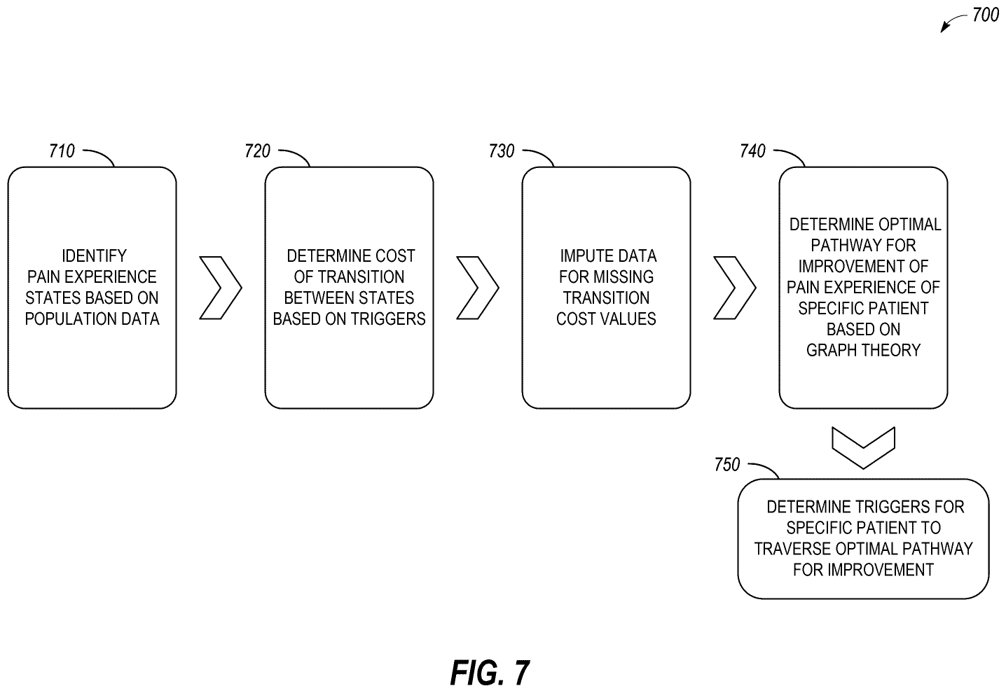

As described in more detail below with respect to FIGS. 7 to 13, a user, e.g., the patient, or a clinician or other medical professional associated with the patient can select, load, modify, and implement one or more parameters of a defined program for neurostimulation treatment, based on logic that identifies different therapeutic programs using a pain treatment modeling system. The programming determination logic can determine which program is likely to produce an improvement for a predetermined chronic pain condition, based on a planned path and state transition to advance the patient towards an optimal/best pain experience state. Example parameters that can be implemented by a selected program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time).

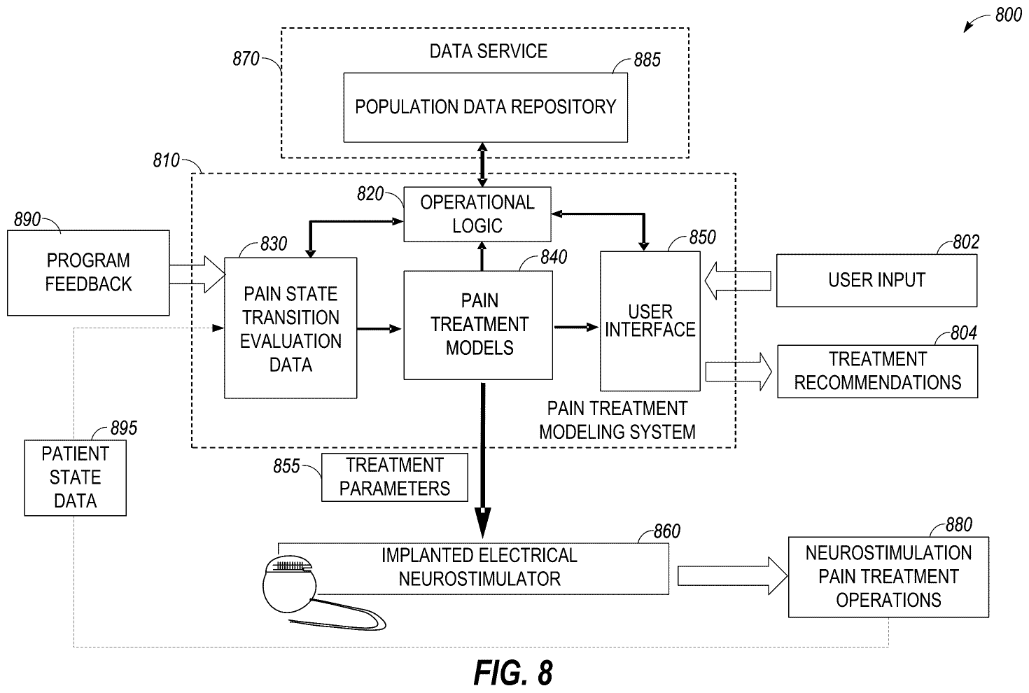

As detailed in FIG. 6, a controller, e.g., controller 650 of FIG. 6, can implement program(s) and parameter setting(s) to implement a specific neurostimulation waveform, pattern, or energy output, using a program or setting in storage, e.g., external storage device 618 of FIG. 6, or using settings communicated via an external communication device 620 of FIG. 6 corresponding to the selected program. The implementation of such program(s) or setting(s) may further define a therapy strength and treatment type corresponding to a specific pulse group, or a specific group of pulse groups, based on the specific program(s) or setting(s). As also described in more detail below with respect to FIG. 8 and thereafter, a pain treatment modeling system and associated logic may operate to select such programs(s) or settings(s) based on suitability for a particular patient in a particular pain experience state. A clinician or the patient may also affect use and implementation of such programs or settings, including in settings where a combination of recommended/automatic and manual control are involved.

Portions of the stimulation device 104, e.g., implantable medical device, or the programming device 102 can be implemented using hardware, software, or any combination of hardware and software. Portions of the stimulation device 104 or the programming device 102 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The system 100 could also include a subcutaneous medical device (e.g., subcutaneous ICD, subcutaneous diagnostic device), wearable medical devices (e.g., patch based sensing device), or other external medical devices.

FIG. 2 illustrates an embodiment of a stimulation device 204 and a lead system 208, such as may be implemented in neurostimulation system 100 of FIG. 1. Stimulation device 204 represents an embodiment of stimulation device 104 and includes a stimulation output circuit 212 and a stimulation control circuit 214. Stimulation output circuit 212 produces and delivers neurostimulation pulses, including the neurostimulation waveform and parameter settings implemented via a program selected or implemented with the user interface 110. Stimulation control circuit 214 controls the delivery of the neurostimulation pulses using the plurality of stimulation parameters, which specifies a pattern of the neurostimulation pulses. Lead system 208 includes one or more leads each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes. Those of ordinary skill in the art will understand that the neurostimulation system 100 may include additional components such as sensing circuitry for patient monitoring and/or feedback control of the therapy, telemetry circuitry, and power.

The neurostimulation system may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. Other parameters that may be controlled or varied include the amplitude, pulse width, and rate (or frequency) of the electrical pulses. Each electrode configuration, along with the electrical pulse parameters, can be referred to as a “modulation parameter” set. Each set of modulation parameters, including fractionalized current distribution to the electrodes (as percentage cathodic current, percentage anodic current, or off), may be stored and combined into a program that can then be used to modulate multiple regions within the patient.

The neurostimulation system may be configured to deliver different electrical fields to achieve a temporal summation of modulation. The electrical fields can be generated respectively on a pulse-by-pulse basis. For example, a first electrical field can be generated by the electrodes (using a first current fractionalization) during a first electrical pulse of the pulsed waveform, a second different electrical field can be generated by the electrodes (using a second different current fractionalization) during a second electrical pulse of the pulsed waveform, a third different electrical field can be generated by the electrodes (using a third different current fractionalization) during a third electrical pulse of the pulsed waveform, a fourth different electrical field can be generated by the electrodes (using a fourth different current fractionalized) during a fourth electrical pulse of the pulsed waveform, and so forth. These electrical fields can be rotated or cycled through multiple times under a timing scheme, where each field is implemented using a timing channel. The electrical fields may be generated at a continuous pulse rate, or as bursts of pulses. Furthermore, the interpulse interval (i.e., the time between adjacent pulses), pulse amplitude, and pulse duration during the electrical field cycles may be uniform or may vary within the electrical field cycle. Some examples are configured to determine a modulation parameter set to create a field shape to provide a broad and uniform modulation field such as may be useful to prime targeted neural tissue with sub-perception modulation. Some examples are configured to determine a modulation parameter set to create a field shape to reduce or minimize modulation of non-targeted tissue (e.g., dorsal column tissue). Various examples disclosed herein may use programming that shapes the modulation field to enhance modulation of some neural structures and diminish modulation at other neural structures. The modulation field may be shaped by using multiple independent current control (MICC) or multiple independent voltage control to guide the estimate of current fractionalization among multiple electrodes and estimate a total amplitude that provide a desired strength. For example, the modulation field may be shaped to enhance the modulation of dorsal horn neural tissue and to minimize the modulation of dorsal column tissue. A benefit of MICC is that MICC accounts for various in electrode-tissue coupling efficiency and perception threshold at each individual contact, so that “hotspot” stimulation is eliminated.

The number of electrodes available combined with the ability to generate a variety of complex electrical pulses, presents a huge selection of available modulation parameter sets to the clinician or patient. For example, if the neurostimulation system to be programmed has sixteen electrodes, millions of modulation parameter value combinations may be available for programming into the neurostimulation system. Furthermore, some SCS systems may have as many as thirty-two electrodes, which exponentially increases the number of modulation parameter value combinations available for programming. To facilitate such programming, a clinician often initially programs and modifies the modulation parameters through a computerized programming system, to allow the modulation parameters to be established from starting parameter sets (programs) and patient and clinician feedback. In addition, the patient often is provided with a limited set of controls to switch from a first program to a second program, based on user preferences and the subjective amount of pain or discomfort that the patient is treating. However, the implementation and use of a pain treatment modeling system and pain state evaluation logic as described further in FIGS. 7 to 13 provides a mechanism for recommending and controlling programming—including the timing and intensity of such programming—to emphasize chronic pain improvement and therapy progression among different pain experience states of a treatment pathway.

FIG. 3 illustrates an embodiment of a programming device 302, such as may be implemented in neurostimulation system 100. Programming device 302 represents an embodiment of programming device 102 and includes a storage device 318, a programming control circuit 316, and a user interface device 310. Programming control circuit 316 generates the plurality of stimulation parameters that controls the delivery of the neurostimulation pulses according to the pattern of the neurostimulation pulses. The user interface device 310 represents an embodiment to implement the user interface 110.

In various embodiments, the user interface device 310 includes an input/output device 320 that is capable to receive user interaction and commands to load, modify, and implement neurostimulation programs and schedule delivery of the neurostimulation programs. In various embodiments, the input/output device 320 allows the user to create, establish, access, and implement respective parameter values of a neurostimulation program through graphical selection (e.g., in a graphical user interface output with the input/output device 320), including values of a therapeutic neurostimulation field. In some examples, the user interface device 310 can receive user input to initiate the implementation of the programs which are recommended, modified, selected, or loaded through use of a pain treatment modeling system, which are described in more detail below.

In various embodiments, the input/output device 320 allows the patient user to apply, change, modify, or discontinue certain building blocks of a program and a frequency at which a selected program is delivered. In various embodiments, the input/output device 320 can allow the patient user to save, retrieve, and modify programs (and program settings) loaded from a clinical encounter, managed from the patient feedback computing device, or stored in storage device 318 as templates. In various embodiments, the input/output device 320 and accompanying software on the user interface device 310 allows newly created building blocks, program components, programs, and program modifications to be saved, stored, or otherwise persisted in storage device 318.

In one embodiment, the input/output device 320 includes a touchscreen. In various embodiments, the input/output device 320 includes any type of presentation device, such as interactive or non-interactive screens, and any type of user input device that allows the user to interact with a user interface to implement, remove, or schedule the programs, and as applicable, to edit or modify waveforms, building blocks, and program components. Thus, the input/output device 320 may include one or more of a touchscreen, keyboard, keypad, touchpad, trackball, joystick, and mouse. In various embodiments, circuits of the neurostimulation system 100, including its various embodiments discussed in this document, may be implemented using a combination of hardware and software. For example, the logic of the user interface 110, the stimulation control circuit 214, and the programming control circuit 316, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

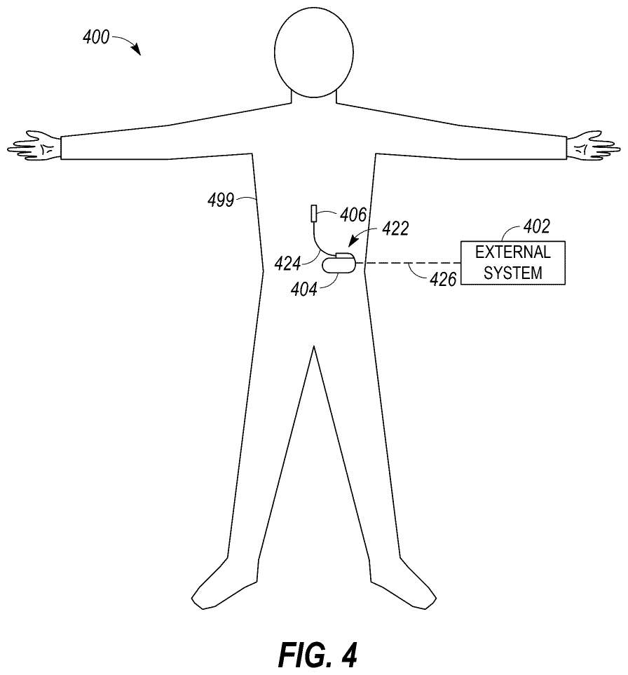

FIG. 4 illustrates an implantable neurostimulation system 400 and portions of an environment in which system 400 may be used. System 400 includes an implantable system 422, an external system 402, and a telemetry link 426 providing for wireless communication between an implantable system 422 and an external system 402. Implantable system 422 is illustrated in FIG. 4 as being implanted in the patient's body 499. The system is illustrated for implantation near the spinal cord. However, the neuromodulation system may be configured to modulate other neural targets.

Implantable system 422 includes an implantable stimulator 404 (also referred to as an implantable pulse generator, or IPG), leads 424 provided by a lead system, and electrodes 406, which represent an embodiment of the stimulation device 204, the lead system 208, and the electrodes 206, respectively. The external system 402 represents an embodiment of the programming device 302.

In various embodiments, the external system 402 includes one or more external (non-implantable) devices each allowing the user and/or the patient to communicate with the implantable system 422. In some embodiments, the external system 402 includes a programming device intended for the user to initialize and adjust settings for the implantable stimulator 404 and a remote control device intended for use by the patient. For example, the remote control device may allow the patient to turn the implantable stimulator 404 on and off and/or adjust certain patient-programmable parameters of the plurality of stimulation parameters. The remote control device may also provide a mechanism to receive and process feedback on the operation of the implantable neuromodulation system. Feedback may include metrics or an efficacy indication reflecting perceived pain, effectiveness of therapies, or other aspects of patient comfort or condition. Such feedback may be automatically detected from a patient's physiological state, or manually obtained from user input entered in a user interface.

For the purposes of this specification, the terms “neurostimulator,” “stimulator,” “neurostimulation,” and “stimulation” generally refer to the delivery of electrical energy that affects the neuronal activity of neural tissue, which may be excitatory or inhibitory; for example by initiating an action potential, inhibiting or blocking the propagation of action potentials, affecting changes in neurotransmitter/neuromodulator release or uptake, and inducing changes in neuro-plasticity or neurogenesis of tissue. It will be understood that other clinical effects and physiological mechanisms may also be provided through use of such stimulation techniques.

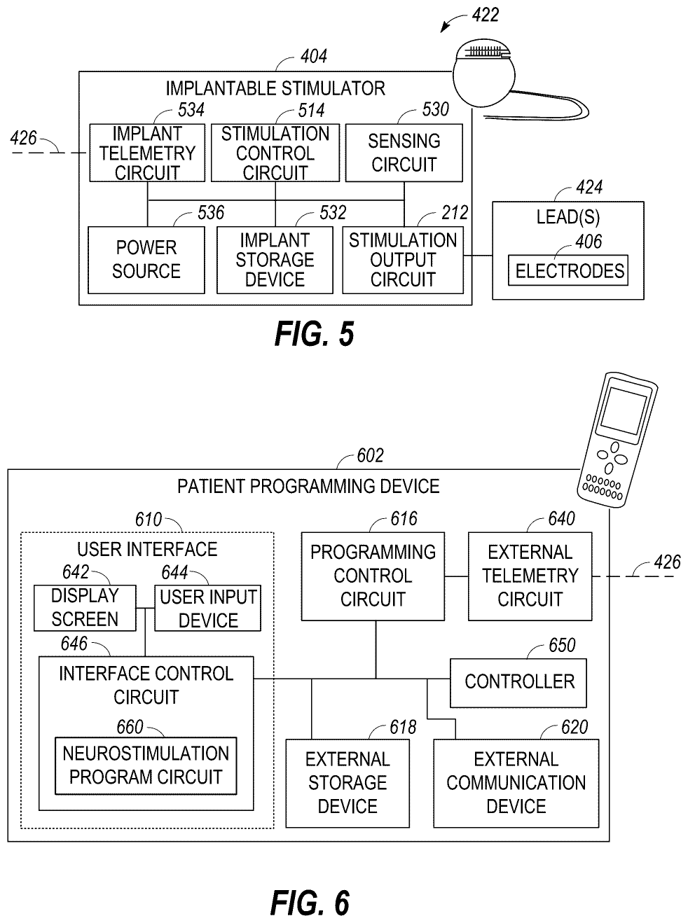

FIG. 5 illustrates an embodiment of the implantable stimulator 404 and the one or more leads 424 of an implantable neurostimulation system, such as the implantable system 422. The implantable stimulator 404 may include a sensing circuit 530 that is optional and required only when the stimulator has a sensing capability, stimulation output circuit 212, a stimulation control circuit 514, an implant storage device 532, an implant telemetry circuit 534, and a power source 536. The sensing circuit 530, when included and needed, senses one or more physiological signals for purposes of patient monitoring and/or feedback control of the neurostimulation. Examples of the one or more physiological signals includes neural and other signals each indicative of a condition of the patient that is treated by the neurostimulation and/or a response of the patient to the delivery of the neurostimulation.

The stimulation output circuit 212 is electrically connected to electrodes 406 through the one or more leads 424, and delivers each of the neurostimulation pulses through a set of electrodes selected from the electrodes 406. The stimulation output circuit 212 can implement, for example, the generating and delivery of a customized neurostimulation waveform (e.g., implemented from a parameter or programming setting selected with the present pain treatment modeling system) to an anatomical target of a patient.

The stimulation control circuit 514 represents an embodiment of the stimulation control circuit 214 and controls the delivery of the neurostimulation pulses using the plurality of stimulation parameters specifying the pattern of the neurostimulation pulses. In one embodiment, the stimulation control circuit 514 controls the delivery of the neurostimulation pulses using the one or more sensed physiological signals and processed input from patient feedback interfaces. The implant telemetry circuit 534 provides the implantable stimulator 404 with wireless communication with another device such as a device of the external system 402, including receiving values of the plurality of stimulation parameters from the external system 402. The implant storage device 532 stores values of the plurality of stimulation parameters, including parameters from one or more programs obtained or selected using the present pain treatment modeling system disclosed herein.

The power source 536 provides the implantable stimulator 404 with energy for its operation. In one embodiment, the power source 536 includes a battery. In one embodiment, the power source 536 includes a rechargeable battery and a battery charging circuit for charging the rechargeable battery. The implant telemetry circuit 534 may also function as a power receiver that receives power transmitted from external system 402 through an inductive couple.

In various embodiments, the sensing circuit 530 (if included), the stimulation output circuit 212, the stimulation control circuit 514, the implant telemetry circuit 534, the implant storage device 532, and the power source 536 are encapsulated in a hermetically sealed implantable housing. In various embodiments, the lead(s) 424 are implanted such that the electrodes 406 are placed on and/or around one or more targets to which the neurostimulation pulses are to be delivered, while the implantable stimulator 404 is subcutaneously implanted and connected to the lead(s) 424 at the time of implantation.

FIG. 6 illustrates an embodiment of an external patient programming device 602 of an implantable neurostimulation system, such as the external system 402, with the external patient programming device 602 illustrated to receive commands (e.g., program selections, information) directly or indirectly from a pain treatment modeling system and the logic associated with pain state transitions and therapy pathways (not shown in FIG. 6, but discussed with reference to FIGS. 7 to 13, below). The external patient programming device 602 represents an embodiment of the programming device 302, and includes an external telemetry circuit 640, an external storage device 618, a programming control circuit 616, a user interface device 610, a controller 650, and an external communication device 620.

The external telemetry circuit 640 provides the external patient programming device 602 with wireless communication to and from another controllable device such as the implantable stimulator 404 via the telemetry link 426, including transmitting one or a plurality of stimulation parameters (including changed stimulation parameters of a newly selected program) to the implantable stimulator 404. In one embodiment, the external telemetry circuit 640 also transmits power to the implantable stimulator 404 through inductive coupling.

The external communication device 620 provides a mechanism to conduct communications with a programming information source, such as a data service, pain treatment modeling system, or other aspects of a dynamic information system, to receive programming information via an external communication link (not shown). As described in the following paragraphs, the pain treatment modeling system may be used to identify a program or program data to the external patient programming device 602 that corresponds to a new or different neurostimulation program or characteristics of a neurostimulation program (which is, in turn, selected to provide an improved or different treatment of a chronic pain condition, along a treatment pathway). The external communication device 620 and the programming information source may communicate using any number of wired or wireless communication mechanisms described in this document, including but not limited to an IEEE 802.11 (Wi-Fi), Bluetooth, Infrared, and like standardized and proprietary wireless communications implementations. Although the external telemetry circuit 640 and the external communication device 620 are depicted as separate components within the external patient programming device 602, the functionality of both of these components may be integrated into a single communication chipset, circuitry, or device.

The external storage device 618 stores a plurality of existing neurostimulation waveforms, including definable waveforms for use as a portion of the pattern of the neurostimulation pulses, settings and setting values, and other portions of a program. In various embodiments, each waveform of the plurality of individually definable waveforms includes one or more pulses of the neurostimulation pulses, and may include one or more other waveforms of the plurality of individually definable waveforms. Examples of such waveforms include pulses, pulse blocks, pulse trains, and train groupings, and programs. The existing waveforms stored in the external storage device 618 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 618 also stores a plurality of individually definable fields that may be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program (including settings changed as a result of pain state evaluation and pain treatment modeling) may be correlated to the control of these waveforms and definable fields.

The programming control circuit 616 represents an embodiment of a programming control circuit 316 and generates the plurality of stimulation parameters, which is to be transmitted to the implantable stimulator 404, based on the pattern of the neurostimulation pulses. The pattern is defined using one or more waveforms selected from the plurality of individually definable waveforms (e.g., defined by a program) stored in an external storage device 618. In various embodiments, a programming control circuit 616 checks values of the plurality of stimulation parameters against safety rules to limit these values within constraints of the safety rules. In one embodiment, the safety rules are heuristic rules.

The user interface device 610 represents an embodiment of the user interface device 310 and allows the user (including a patient, caregiver, or clinician) to select, modify, enable, disable, activate, schedule, or otherwise define a program or sets of programs for use with the neurostimulation device and perform various other monitoring and programming tasks for operation of the neurostimulation device. The user interface device 610 can enable a user to implement, save, persist, or update a program including the program or program parameters recommended or indicated by the programming information source, such as a data service or pain treatment modeling system. The user interface device 610 includes a display screen 642, a user input device 644, and an interface control circuit 646. The display screen 642 may include any type of interactive or non-interactive screens, and the user input device 644 may include any type of user input device that supports the various functions discussed in this document, such as a touchscreen, keyboard, keypad, touchpad, trackball, joystick, and mouse. The user interface device 610 may also allow the user to perform any other functions discussed in this document where user interface input is suitable.

Interface control circuit 646 controls the operation of the user interface device 610 including responding to various inputs received by the user input device 644 that define or modify characteristics of implementation (including conditions, schedules, and variations) of one or more programs, parameters within the program, characteristics of one or more stimulation waveforms within a program, and like neurostimulator operational values that may be entered or selected with the external patient programming device 602, or obtained from the programming information source, such as the data service, or the pain treatment modeling system. Interface control circuit 646 includes a neurostimulation program circuit 660 that may generate a visualization of such characteristics of implementation, and receive and implement commands to implement the program and the neurostimulator operational values (including a status of implementation for such operational values). These commands and visualization may be performed in a review and guidance mode, status mode, or in a real-time programming mode.

The controller 650 can be a microprocessor that communicates with the external telemetry circuit 640, the external communication device 620, the external storage device 618, the programming control circuit 616, and the user interface device 610, via a bidirectional data bus. The controller 650 can be implemented by other types of logic circuitry (e.g., discrete components or programmable logic arrays) using a state machine type of design. As used in this disclosure, the term “circuitry” should be taken to refer to either discrete logic circuitry, firmware, or to the programming of a microprocessor.

As will be understood, the variety of settings for a neurostimulation device may be provided by many variations of programming parameter settings within programs. Existing patient programmers only provide a limited ability for a patient to cycle through programs that have defined programming parameters, with hundreds or thousands of specific settings often being rolled up into a single program. The following system and methods provide technical mechanisms to generate and recommend new programs and parameters for chronic pain therapy in response to planned guidance and transitions along a pathway that has multiple pain experience states.

Newer approaches of neurostimulation involve controlling systems and controllers with a feedback control mechanism, to provide changes and adaptation based on a patient's local tissue response or self-reported pain level. However, the best pathway that a patient can take through their healing journey is not necessarily the shortest. Often, the high initial “cost” of transitioning to an improved pain state can be demoralizing, and thus can hamper overall progress of treatment. In addition, the best pain state that can be reached as a result of ongoing neurostimulation therapy can vary between patients or be completely inaccessible for others.

The following approaches consider population-based measures of pain experience transition costs—in addition to individualized measures such as patient goals and psychology—in order to recommend the best pathway to an improved/optimized pain experience for a patient. Here, the “costs” of transitioning between pain states refers to how hard it is for a patient to achieve that next state. The approaches below enable an approach for determining pathways of different states in a treatment progression, determining the costs of transition among different states, and evaluating progress relative to overall pain experience states, to consider not only pain but also related quality of life aspects.

FIG. 7 depicts an overview of sequential operations 700 for evaluating pain experience states in connection with neurostimulation therapy. At a high level, the operations 700 depict how a pain treatment modeling system will analyze patient population data to produce useful data states and predictions applicable to a single, specific patient. The examples below refer to a single “individual” or “specific” patient, but the examples are applicable to more than one patient (e.g., groups or categories of patients) with similar conditions or symptoms.

Operation 710 depicts the identification of pain experience states based on population data. For instance, the different pain experience states of a given a patient population can be identified using clustering data from available and relevant daily signals. Pain experience states can be identified using several clinical variables identified from a sufficiently large cohort (e.g., with N>100). Clinical variables relevant to a pain experience state may include: self-reported pain level; medications used (e.g., use of opioids, over-the-counter pain relievers including NSAID medications, sleep mediations); self-reported activity level; objective activity or fitness level collected from sensors of a wearable device (e.g., smart watch, smart ring); self-reported mental states (e.g., mood, alertness); and other physiological-related measurements.

Operation 720 depicts a determination of the cost of transition between pain experience states, based on triggers. This determination may include the use of longitudinal patient data to build a state transition matrix for each subject of a patient population. The costs of transition may be determined or estimated based on triggers associated with changes or transitions between known pain experience states. In some examples, a cost of transition may be associated with a penalty, based on a particular characteristic or known limitation of a respective subject.

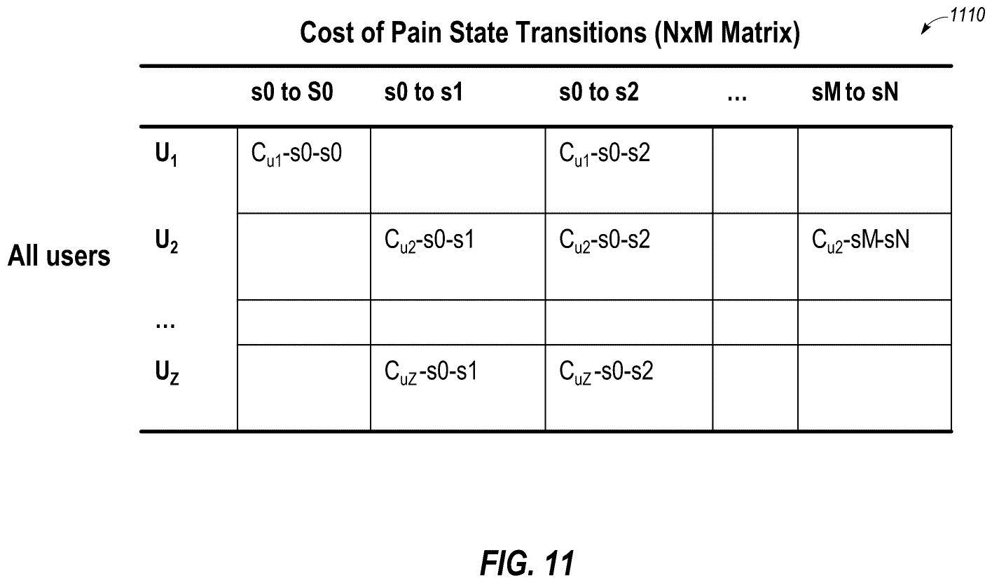

Operation 730 depicts the imputation of data for missing transition cost values, for each subject of a patient population. For example, missing data in a population data set can be inferred using a collaborative filtering approach. Because not all subjects will experience each and every pain experience state, data for the cost of transition between states can be imputed based on one or more approaches, including the behavioral analysis of subjects. Details of an example approach for imputing missing data are discussed below with reference to FIG. 11.

Operation 740 includes building a personalized pain state pathway for a particular patient to undergo neurostimulation therapy. Patient goals and resiliency can be determined for the particular patient based on standard questionnaires (e.g., a pain catastrophizing scale, or a fear avoidance scale). Here, resilience or resiliency refers to the ability (and likelihood) of a patient to continue with—and in some cases, endure—a transition to another state with a particular use of neurostimulation therapy and medication, even if the transition involves difficult or challenging changes. After all the costs are identified, graph theory is used to determine the “optimal” pathway for health improvement of the particular patient.

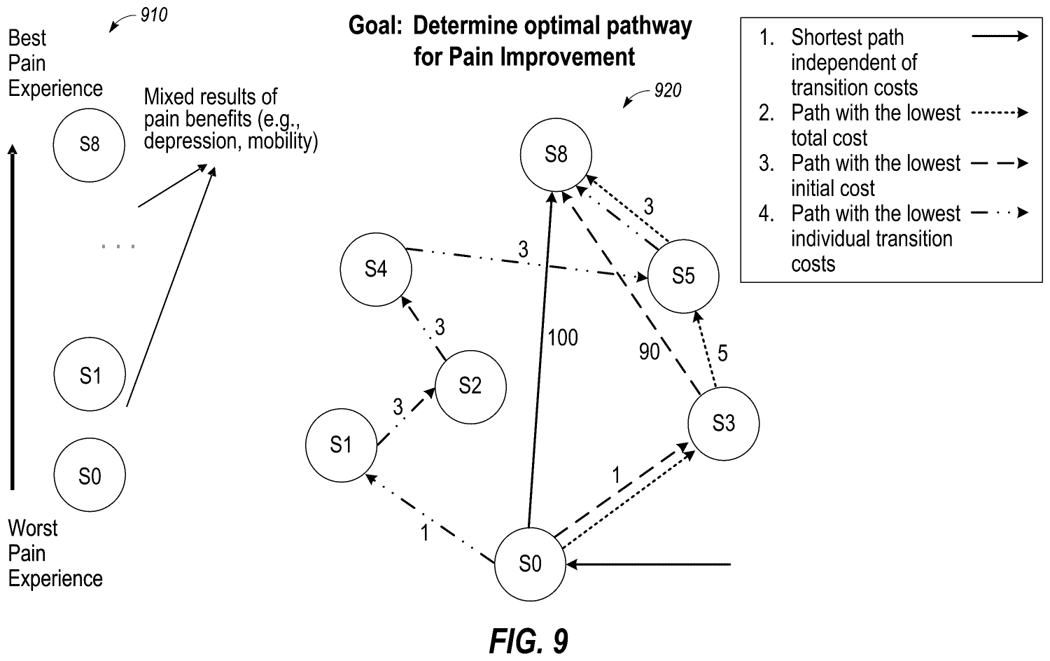

In an example, the pathway for a patient may be selected based on iterating and evaluating the following strategies: 1) a shortest path to a final, optimal pain experience state independent of transition costs; 2) a path with the lowest total cost to reach the final, optimal pain experience state; 3) a path with the lowest initial cost to reach the final, optimal pain experience state; 4) a path with the lowest individual transition costs to reach the final, optimal pain experience state. Separate neurostimulation control programs or controller programming may be associated with each path, or with individual states along each path. For instance, a controller may be configured to select or recommend programs (e.g., electrical neuromodulation signals of different patterns and intensity) that are best suited to achieve the next pain experience state along the path with the lowest individual transition costs. The selection and traversal of this pathway is discussed below with reference to FIG. 9.

Operation 750 includes determining triggers for the specific patient to traverse an optimal pathway for improvement. This includes determining what settings are most appropriate to activate at which time for the particular patient. Then, once a pathway is determined, programming operations can determine or activate all the triggers needed to enable a patient to pass through that optimal pathway. This optimal pathway may include not only the use of specific neurostimulation programming actions, but also other actions relating to the treatment of pain (such as, for instance, an increase in over-the-counter pain medication to advance from a particular state to another state).

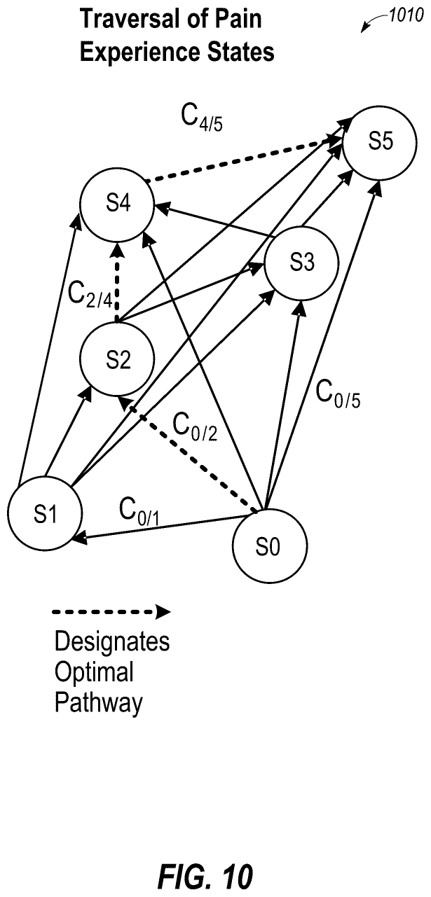

FIG. 8 illustrates, by way of example, a block diagram 800 of an embodiment of a pain treatment modeling system 810, implemented as a computerized system for evaluating and recommending programming parameters of a neurostimulation therapy based on pain experience states and pathways. As shown, the block diagram 800 illustrates data flows among a data service 870, the pain treatment modeling system 810, and a neurostimulation device 860. It will be understood that additional data flows and executed logic operations may occur in connection with the use of the pain treatment modeling system 810 to implement the pathway planning and state evaluation operations discussed below with reference to FIGS. 9 and 10.

The pain treatment modeling system 810 may receive input from a population of human subjects (other patients), such as in the form of longitudinal clinical data such as demographics and self-assessment (e.g., mood) provided in a population data repository 885 of a data service 870. In an example, the operational logic 820 of the pain treatment modeling system 810 accesses and retrieves/obtains historical data relevant to neurostimulation treatment and outcomes in the population subjects. The pain treatment modeling system 810 may use operational logic 820 to perform data processing operations on this population data including factorization, imputations with collaborative filtering, and graph theory. The operational logic 820 may perform the data processing operations to determine pain experience states, state transitions, and pathways for chronic pain treatments experienced by various members of the population.

The pain treatment modeling system 810 uses this information (derived from the population data) to determine relevant pain experience states and possible therapy pathways for a particular human patient. The pain treatment modeling system 810 may evaluate this data to determine an optimal sequence of actions and events (referred to as a path, pathway, or trajectory) for each subject, and identify a particular neurostimulation controller, program, set of programs, or programming to produce treatment parameters 855 associated with a particular pain experience state. Further, the pain treatment modeling system 810 may associate this information with data of one or more pain treatment models 840 to identify an appropriate set of treatment parameters 855, and then provide a selection or recommendation of associated programs that include the treatment parameters 855. The selection or recommendation of the programs (or programming parameters) may be specifically based on possible pathways to traverse pain experience states and reach some goal state, while considering the transition costs/difficulty of these pathways, as discussed below.

The pain states and pain state transitions may be represented with the use of pain state transition evaluation data 830. In an example, pain state transition evaluation data 830 includes data obtained or derived from the ongoing operation of the neurostimulation device 860, including respective pain treatment operations 880, and resulting patient state data 895 or program feedback 890. As a result, the pain treatment modeling system 810 can track what state the patient is at, what next state is appropriate for the patient, and whether particular treatment operations are working. The operational logic 820 may also receive patient state information from user input 802 (e.g., input via the user interface 850) that provides relevant data parameters and patient characteristics for evaluation. The pain treatment modeling system 810 can generate additional treatment recommendations 804 (e.g., to be provided through the user interface 850), or automatically generate or select a program with relevant treatment parameters 855, for control of the implanted neurostimulation device 860.

The operation of the neurostimulation device 860 with the selected program and parameters results in pain treatment operations 880 for chronic pain relief. The results of the pain treatment operations 880, program feedback 890, patient state data 895, or operation of the neurostimulation device 860, can provide learning data that reinforces (emphasizes, de-emphasizes) certain outcomes or treatment operations. The user interface 850 may also receive additional settings (not shown), constraints, and conditions for operation and use of individual models or selections of particular pain experience states, for a particular patient. The pain treatment modeling system 810 may also output other types of treatment recommendations 804 associated with the neurostimulation therapy, such as general health recommendations with instructions for the patient, a clinician, a caregiver, etc.

The pain treatment modeling system 810 shown in FIG. 8 may be implemented in the form of a computing device (e.g., a server) with the computing device being specially programmed to communicate over a network the results of the operational logic 820 (e.g., with an algorithm implemented in software that is executed on the computing device) or the selection of the treatment parameters 855. It will be understood that other form factors and embodiments of the pain treatment modeling system 810, including in the integration of other programming devices, data services, or information services, may also be provided. In some examples, the data service 870 and the population data repository 885 may be operated or hosted by a research institution, medical service provider, or a medical device provider (e.g., a manufacturer of the neurostimulation device) that collects and analyzes population data. The data service 870 may be accessed using an application programming interface (API) or other remotely accessible interface accessible via the network. In other examples, the data service 870 and the pain treatment modeling system 810 are integrated into a same system.

FIG. 9 depicts pain experience states, pain experience state transitions, and pain improvement pathways evaluated in connection with a neurostimulation therapy. The pain experience states are shown in a ranked set of pain experience states 910 and a graph of possible pathways 920. This figure graphically depicts some of the complexities that are considered for planning a treatment pathway for a patient who will encounter different pain experiences during a therapy regimen. While a patient may desire to proceed directly to a “best” pain experience state, the cost of doing so may be extremely high—if not impossible.

To demonstrate this, FIG. 9 depicts the ranked set of pain experience states 910, ranked from state S0 (the “worst” or “starting” pain experience state) to state S8 (the “best” or “goal” pain experience state), with six intermediate states not depicted for purposes of simplicity. The state S0 generally corresponds to a state with the highest amount of pain, and the state S8 generally corresponds to a state with the lowest amount of pain. However, each of the states are also associated with other mixed results of pain management. For instance, a better (higher numbered) state may experience decreased pain but with the tradeoff of increased depression or mood effects, or decreased mobility. Or, a better state may provide an increased duration of pain, but at a decreased intensity (less sharp pain) with other effects such as better sleep. The state S8 generally corresponds to an end goal with a “best” outcome, whereas the state S0 generally corresponds to a starting position with a “worst” outcome. However, it will be understood that the user may start at some intermediate location, and not all states may be encountered or experienced by the patient.

Additionally, FIG. 9 depicts the graph of possible pathways 920 for traversal from state SO to S8, including labels that identify transition costs between available states. The possible pathways 920 also includes a designation of: (1) a shortest path independent of transition costs; (2) a path with the lowest total cost; (3) a path with the lowest initial cost; and (4) a path with the lowest individual transition costs. The shortest path independent of transition costs is shown from state S0 to state S8, with a transition cost of 100 (the highest total cost, 100 out of a maximum 100). The path with the lowest total cost is shown as the pathway from S0->S3->S5->S8 (a lowest total cost of 9). The path with the lowest initial cost is shown as the pathway from S0->S3->S8 (a total cost of 91, because the transition cost from S3->S8 is 90 although the initial cost from S0->S3 is only 1). The path with the lowest individual transition costs is shown from state S0->S1->S2->S4->S5->S8 (a total cost of 13). This path with the lowest individual transition costs can provide a suitable pathway for a patient who has low resilience and needs a more gradual path of treatment changes.

FIG. 10 depicts a mapping of pain state transitions and pathways on a graph 1010 in connection with a neurostimulation therapy. Here, cost values are labeled on the graph 1010 to represent the costs between different states (e.g., C_{0/1} referring to the costs of transitioning from S0 to S1, C_{0/2} referring to the costs of transitioning from S0 to S2, and so on). These cost values can be associated with particular data table entries as discussed below with reference to FIG. 11.

For example, consider a scenario where a chronic pain patient uses a Spinal Cord Stimulator (SCS), and the patient's pain state is evaluated on different dimensions (e.g., mood, pain, sleep, medication, etc.) at the initial patient visit. The SCS device includes multiple stimulation programs that the patient can choose from to relieve their symptoms. Using the data collected about this patient, a pain state is assigned to the current condition of the subject using a previously established model of pain state assessment. For this use case, the model assigns one out of a total of six pain experience states (S0 to S5). For the next month, the data is collected daily and for each day the corresponding pain state is assigned. By observing the frequency of state transitions, the costs of transitioning from any two states are calculated. If transitions from any two states do not exist, a maximal default value is assigned for that transition.

The pain treatment modeling system can recommend one or more programs for the SCS device based on programs or controller logic associated with at least four different types of approaches. These approaches may be selected from the paths discussed above, e.g.: Path 1: Shortest path independent of transition costs; Path 2: Path with the lowest total cost; Path 3: Path with the lowest initial cost; Path 4: Path with the lowest individual transition costs.

Next, the patient completes a Pain Resilience Scale (PRS) questionnaire and has an assigned PRS score. The PRS score shows that the patient has very low resilience. Based on the PRS result, the patient is advised to use a modeling approach that corresponds to Path 4, the path with the lowest individual transition costs. A corresponding sequence of neurostimulator programs and programming settings can then be applied to provide recommended settings, and automatically transition the patient between states with the lowest individual transition costs.

Accordingly, based on these states and costs, a pain treatment modeling system may provide a selection of particular parameter(s) or program(s) for a neurostimulation device of a particular patient. In some examples, the pain treatment modeling system may automatically select or construct an entirely new program or may customize or modify a program or program settings based on pain experience states. Other patient or clinician user interfaces and program implementation logic may be used to activate, deploy, select, define, edit, and modify the parameter(s) or program(s) based on the specific characteristics of a patient or the selected transition path for the patient.

FIG. 11 depicts a matrix 1110 of data values from multiple users in connection with the evaluation of pain state transitions. In some examples, all (or nearly all) costs associated with transitions are needed to compute the optimal trajectory. However, in any training dataset provided from real-world population data, not all state transitions will be known for each user. As a result, an imputation method can be used to determine missing cost data values.

In one example, a matrix is built to impute cost for unknown transitions from users, such as in the format of the matrix 1110. The matrix 1110 is depicted as tracking cost data values where one dimension (the Y-dimension in matrix 1110) corresponds to the number of users and the other dimension (the X-dimension in matrix 1110) is the cost of each possible transition. The matrix 1110 depicted in FIG. 11 shows a number of transition cost values for each user U₁ to U_{Z}, but with many transition cost values missing. In one example, a Singular Value Decomposition (SVD)-based collaborative filtering approach is applied to determine the missing cost values to complete the matrix 1110.

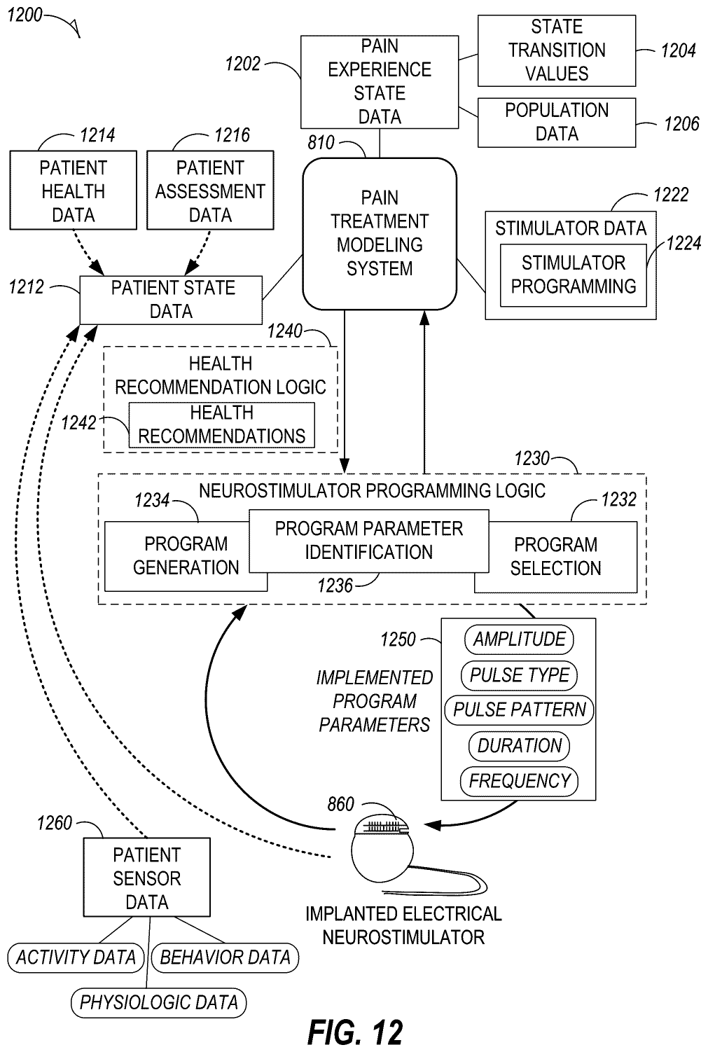

FIG. 12 illustrates, by way of example, an embodiment of a system for evaluating and recommending programming parameters of a neurostimulation therapy based on pain experience states. This figure depicts an embodiment of the pain treatment modeling system 810 that is configured to identify programs and programming parameters of a neurostimulation treatment (e.g., to be implemented for the neurostimulation device 860).

As illustrated, the data inputs for the pain treatment modeling system 810 include pain experience state data 1202, which may include a combination of state transition values 1204 and population data values 1206. The population data values 1206 can provide information relevant to multiple pain experience states and associated conditions for a population of subjects (e.g., other patients), and the state transition values 1204 can provide information related to the transitions among the pain experience states (including the cost, conditions, requirements of transitions, etc.).

The pain treatment modeling system 810 receives data inputs provided from stimulator data 1222, such as in the form of stimulator programming 1224, to determine which type of programming (programs, programming parameter combinations, etc.) is applicable to a particular pain experience state for a particular patient. For instance, the stimulator data 1222 may associate the use of particular stimulator programming 1224 with the use of specific patient conditions and therapy effects. The pain treatment modeling system 810 also receives patient state data 1212 that is specific to the patient to be treated. This patient state data 1212 may include patient health data 1214 provided from objective measurements (e.g., health records) and patient assessment data 1216 provided from subjective measurements (e.g., surveys, questionnaires, etc.). In some examples, the patient state data 1212 (including derived measurements of patient pain states) may be determined from various forms and types of patient sensor data 1260, such as activity data, behavior data, or physiologic data. For instance, physiological data may include data values such as autonomic tone, heart rate, respiratory rate, or blood pressure. Activity data may include data values related to movement or gait. Behavior data may include data values related to a psychological state.

The pain treatment modeling system 810 determines and evaluates the pain experience state of a particular patient, to control neurostimulation effects via neurostimulator programming logic 1220. The neurostimulator programming logic 1220 may identify and affect the use of specific programming parameters 1250 (e.g., treatment parameters) based on the pain experience states, transitions, and pathways, for a patient chronic pain condition to be treated using the neurostimulation device 860. In various examples, the neurostimulator programming logic 1230 may perform program selection 1232, program generation 1234, or program parameter identification 1236, to generate the programming parameters 1250.

As illustrated, the programming parameters 1250 may include defined aspects such as amplitude, pulse type, pulse pattern, duration, and frequency, among other aspects described herein. In an example, the parameters of the stimulator input include a set of predefined stimulator programs, which may include previously generated programs that have results that correspond to a particular patient pain state or experience. Other program types and feedback on these various programs may be collected and considered for a particular patient.

In some examples, the pain treatment modeling system 810 may operate health recommendation logic 1240 to provide health recommendations 1242 that accompany the control of neurostimulation treatment. For instance, the health recommendations 1242 may relate to medication usage, activity, and the control of other medical devices, which have an effect on the patient chronic pain condition or related second-order effects.

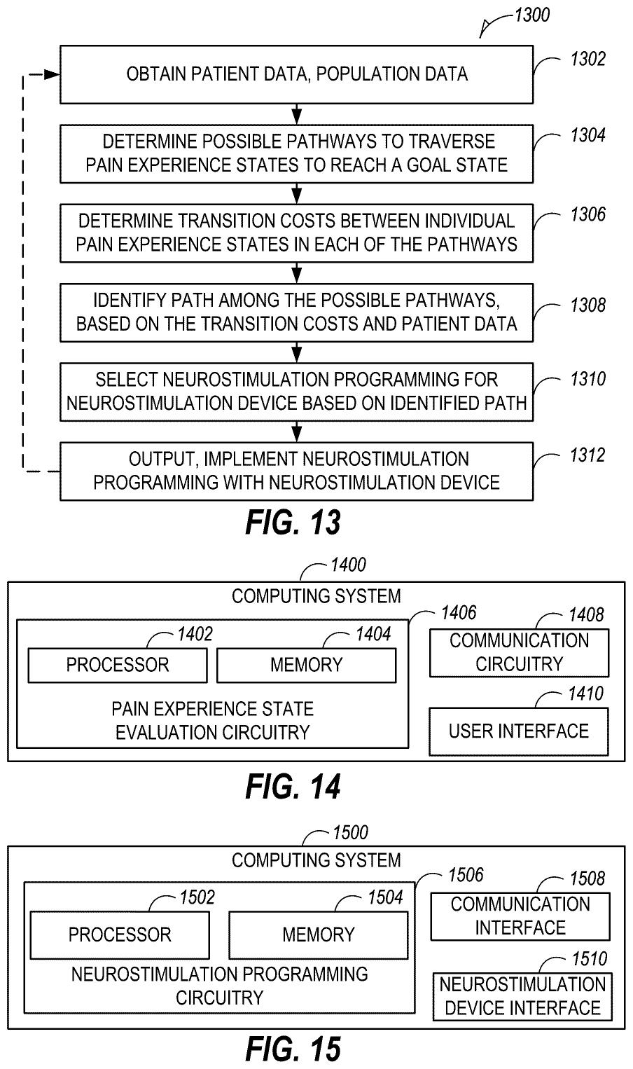

FIG. 13 illustrates, by way of example, an embodiment of a processing method 1300 implemented by a system or device to determine and/or establish programming of an implantable electrical neurostimulation device for treating chronic pain of a human subject. For example, the processing method 1300 can be embodied by operations performed by one or more computing systems or devices that are specially programmed to implement the pain determination, program modeling, and neurostimulation programming functions described herein.

For example, the method 1300 includes operation 1302 to obtain patient data (such as patient state data 895 or patient state data 1212, associated with a particular human patient) and population data (such as population data values 1206, associated with a population of other human patients). The population data may be obtained in connection with the data service 870 and a population data repository 885, as discussed above. The patient data may be obtained in connection with feedback or user inputs, as discussed above.

The method 1300 continues, at operation 1304, to determine possible pathways to traverse pain experience states of the chronic pain condition. In this setting, the possible pathways include respective paths among the pain experience states from a starting state, to one or more intermediate states, to a goal state (e.g., as shown in FIG. 9 and FIG. 10, discussed above). This goal state may be a final or optimal state that is associated with one or more attributes or characteristics that provide an improvement in the chronic pain (e.g., pain level measurement or a pain type measurement) or a condition related to the chronic pain, such as one or more of: a sleep state, a mobility state, a medication state, or an emotional state.

In some examples, operations to determine the possible pathways includes to identify the pain experience states and relative rankings of the pain experience states, to enable the relative rankings to be customized to the human patient. For instance, the relative rankings of the pain experience states may be customized to the human patient based on a measurement of one or more preference associated with the human patient, or a measurement of resilience of the human patient.

The method 1300 continues, at operation 1306, to determine transition costs between the pain experience states involved in each of the possible pathways. For instance, the respective states of the pain experience states are associated with different pain management characteristics based on therapy with the neurostimulation device. These pain experience states may be determined based on the analysis and results of pain experience data collected from a population of patients. The pain experience states may be determined from, and/or associated with, defined attributes corresponding to one or more of: medication management, pain level, emotional state, or mobility.

The method 1300 continues, at operation 1308, to identify a path of the possible pathways to reach the goal state, based on the transition costs and characteristics of the human patient. The transition costs may also be determined based on the analysis and results of pain experience data collected from a population of patients, such as based on a frequency of transitions associated with outcomes in the population of patients.

Consistent with the examples of path evaluation discussed above, identifying the path to reach the goal state for the human patient can include use of one or more path traversal strategy. This path traversal strategy may be provided from one of: a shortest path independent of the transition costs from the starting state to the goal state; a path with a lowest total cost of the transition costs to traverse from the starting state to the goal state; a path with a lowest initial cost of the transition costs to traverse from the starting state to a first of the one or more intermediate states; or a path with a lowest individual transition costs to traverse from the starting state to the one or more intermediate states to the goal state. In this context, the path traversal strategy may be selected for the human patient based on one or more preference associated with the human patient or resilience associated with the human patient.

The method 1300 continues, at operation 1310, to select programming parameters for use in the neurostimulation device of the human patient to cause a neurostimulation therapy, based on the identified path to achieve the goal state. For instance, to select the programming parameters for use in the neurostimulation device of the human patient may include a selection or recommendation of one or more program that includes the programming parameters. The selection or recommendation of different programs may be used in a scenario where each of the respective states used in the possible pathways is associated with a separate program (to be used for the neurostimulation device) that includes respective combinations of the programming parameters.

The method 1300 concludes, at operation 1312, to output and implement neurostimulation programming with the neurostimulation device of the human patient. In this context, deployment of the programming parameters via one or more neurostimulation program or programming modes causes a change in operation of the implantable electrical neurostimulation device. The change of operation may relate to one or more of: pulse patterns, pulse shapes, a spatial location of pulses, waveform shapes, or a spatial location of waveform shapes, for modulated energy provided with a plurality of leads of the implantable electrical neurostimulation device. The types of therapy provided with the neurostimulation may include electrical spinal cord stimulation, an electrical brain stimulation, or an electrical peripheral nerve stimulation.

In an example, the selected neurostimulation program causes a change in operation of the implantable electrical neurostimulation device, with to provide a treatment change corresponding to an expected pain experience state for the human subject. Also in an example, deployment of the neurostimulation programming parameters with the neurostimulation program causes a change in operation of the implantable electrical neurostimulation device, which corresponds to an observed change in a measured value. Also in an example, the generated neurostimulation program specifies a change in programming for the implantable electrical neurostimulation device, with the identified parameters, for one or more of: pulse patterns, pulse shapes, a spatial location of pulses, waveform shapes, or a spatial location of waveform shapes, for modulated energy provided with a plurality of leads of the implantable electrical neurostimulation device.

The method 1300 may also include additional operations (not depicted) that implement or modify the programming of the neurostimulation device with a new or modified program or parameters, or recommendations provided in connection with a new or modified program or parameters. Such programming may be implemented in other manners described above, including with variations involving the use of patient, clinician, or administrator involvement. In some examples, the method 1300 may repeat with a closed or partially-closed loop process of repeating operations at each pain evaluation state. This may include obtaining a new set of patient data at operation 1302 to determine if therapy has progressed the patient to another pain experience state, comparing the new set of patient data to the population data, and determining a new goal state or a new pathway to the existing goal state. The remaining operations 1304-1312 may also be repeated to implement updated programming based on the new goal state or new pathway to the existing goal state. Other variations may be provided based on the examples above.

FIG. 14 illustrates, by way of example, a block diagram of an embodiment of a system 1400 (e.g., a computing system) implementing pain state evaluation circuitry for use to establish programming of an implantable electrical neurostimulation device for treating chronic pain of a human subject. The system 1400 may be a remote control device, patient programmer device, clinician programmer device, pain treatment modeling system, or other external device, usable for the determination of a pain state in connection with pain experience states, state transitions, and treatment pathways discussed herein. In some examples, the system 1400 may be a networked device connected via a network (or combination of networks) to a programming device or programming service using a communication interface 1408, with the programming device or programming service providing output content for the graphical user interface or responding to input of the graphical user interface. The network may include local, short-range, or long-range networks, such as Bluetooth, cellular, IEEE 802.14 (Wi-Fi), or other wired or wireless networks.

The system 1400 includes a processor 1402 and a memory 1404, which can be optionally included as part of pain state determination circuitry 1406. The processor 1402 may be any single processor or group of processors that act cooperatively. The memory 1404 may be any type of memory, including volatile or non-volatile memory. The memory 1404 may include instructions, which when executed by the processor 1402, cause the processor 1402 to implement the features of the user interface, or to enable other features of the pain state evaluation circuitry 1406. Thus, electronic operations in the system 1400 may be performed by the processor 1402 or the circuitry 1406.

For example, the processor 1402 or circuitry 1406 may implement any of the features of the method 1300 (including operations 1302, 1304, 1306, 1308) that analyze data related to a pain experience state, to identify a therapy pathway towards a goal state based on transition costs and patient data, and determine neurostimulation parameters based on the identified pathway. The processor 1402 or circuitry 1406 may implement or select neurostimulation programs, based on the determined programming parameters and related constraints, rules, settings, or preferences; and the processor 1402 or circuitry 1406 may further evaluate or compare the benefit of any implemented neurostimulation program and suitability to a particular patient. The system 1400 may save, output, or cause implementation of this new neurostimulation program, directly or indirectly (e.g., via a programming device or system that in turn communicates and implements the new program to the neurostimulation device). It will be understood that the processor 1402 or circuitry 1406 may also implement other aspects of the logic and processing described above with reference to FIGS. 7-13.

FIG. 15 illustrates, by way of example, a block diagram of an embodiment of a system 1200 (e.g., a computing system) for use to provide (e.g., establish, identify, select, communicate, implement, etc.) programming of an implantable electrical neurostimulation device for treating chronic pain of a human subject. The system 1500 may be operated by a clinician, a patient, a caregiver, a medical facility, a research institution, a medical device manufacturer or distributor, and embodied in a number of different computing platforms. The system 1500 may be a remote control device, patient programmer device, pain treatment modeling system, or other external device, including a regulated device used to directly implement programming commands and modification with a neurostimulation device. In some examples, the system 1500 may be a networked device connected via a network (or combination of networks) to a computing system operating a user interface computing system using a communication interface 1508. The network may include local, short-range, or long-range networks, such as Bluetooth, cellular, IEEE 802.11 (Wi-Fi), or other wired or wireless networks.

The system 1500 includes a processor 1502 and a memory 1504, which can be optionally included as part of neurostimulation programming circuitry 1506. The processor 1502 may be any single processor or group of processors that act cooperatively. The memory 1504 may be any type of memory, including volatile or non-volatile memory. The memory 1504 may include instructions, which when executed by the processor 1502, cause the processor 1502 to implement the features of a programming device, or to enable other features of the neurostimulation programming circuitry 1506 that cause neurostimulation programming. Thus, the following references to electronic operations in the system 1500 may be performed by the processor 1502 or the circuitry 1506.

For example, the processor 1502 or circuitry 1506 may implement any of the features of the method 1300 (including operations 1310, 1312) to establish a neurostimulation program, transmit the program to the neurostimulation device, implement (e.g., save, persist, activate, control) the program in the neurostimulation device, and receive feedback for use of the neurostimulation program, including with use of a neurostimulation device interface 1510. The processor 1502 or circuitry 1506 may further provide data and commands to assist the processing and implementation of the programming using communication interface 1508. It will be understood that the processor 1502 or circuitry 1506 may also implement other aspects of the programming devices and device interfaces described above with reference to FIGS. 7-13.

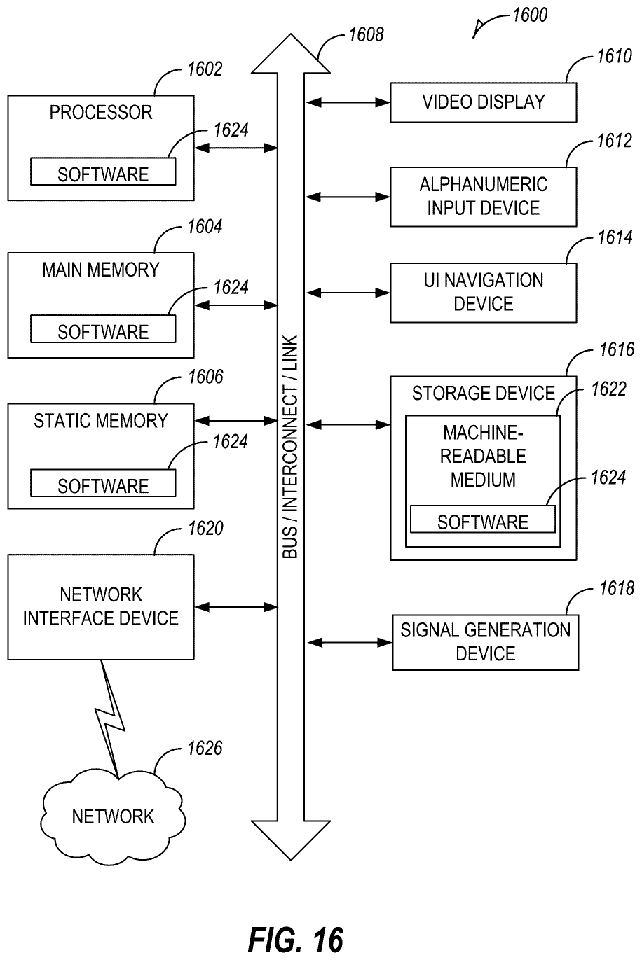

FIG. 16 is a block diagram illustrating a machine in the example form of a computer system 1600, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1600 includes at least one processor 1602 (e.g., a central processing unit (CPU), a graphics processing unit (GPU) or both, processor cores, compute nodes, etc.), a main memory 1604 and a static memory 1606, which communicate with each other via a link 1608 (e.g., bus). The computer system 1600 may further include a video display unit 1610, an alphanumeric input device 1612 (e.g., a keyboard), and a user interface (UI) navigation device 1614 (e.g., a mouse). In one embodiment, the video display unit 1610, input device 1612 and UI navigation device 1614 are incorporated into a touch screen display. The computer system 1600 may additionally include a storage device 1616 (e.g., a drive unit), a signal generation device 1618 (e.g., a speaker), a network interface device 1620, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 16 (such as a GPU, video display unit, keyboard, etc.).

The storage device 1616 includes a machine-readable medium 1622 on which is stored one or more sets of data structures and instructions 1624 (e.g., software) embodying or utilized by any one or more of the methodologies or functions described herein. The instructions 1624 may also reside, completely or at least partially, within the main memory 1604, static memory 1606, and/or within the processor 1602 during execution thereof by the computer system 1600, with the main memory 1604, static memory 1606, and the processor 1602 also constituting machine-readable media.

While the machine-readable medium 1622 is illustrated in an example embodiment to be a single medium, the term “machine-readable medium” may include a single medium or multiple media (e.g., a centralized or distributed database, and/or associated caches and servers) that store the one or more instructions 1624. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1624 may further be transmitted or received over a communications network 1626 using a transmission medium via the network interface device 1620 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A method, performed by a computing device to determine programming of an implantable electrical neurostimulation device for treating a chronic pain condition of a human patient, the method comprising:
determining possible pathways to traverse pain experience states of the chronic pain condition, wherein the possible pathways provide respective paths among the pain experience states from a starting state to one or more intermediate states to a goal state;
determining transition costs between the pain experience states involved in each of the possible pathways, wherein respective states of the pain experience states are associated with different pain management characteristics based on therapy with the neurostimulation device;
identifying a path of the possible pathways to reach the goal state, based on the transition costs and characteristics of the human patient; and
selecting programming parameters for use in the neurostimulation device of the human patient to cause a neurostimulation therapy, based on the identified path to achieve the goal state.

2. The method of claim 1, wherein the pain experience states are determined based on pain experience data collected from a population of patients, and wherein the transition costs are determined based on a frequency of transitions associated with the population of patients.

3. The method of claim 1, wherein determining the possible pathways includes identifying the pain experience states and relative rankings of the pain experience states, and wherein the relative rankings are customized to the human patient.

4. The method of claim 3, wherein the relative rankings are customized to the human patient based on a measurement of: one or more preference associated with the human patient, or a resilience of the human patient.

5. The method of claim 1, wherein identifying the path to reach the goal state for the human patient includes use of a path traversal strategy provided from one of:
a shortest path independent of the transition costs from the starting state to the goal state;
a path with a lowest total cost of the transition costs to traverse from the starting state to the goal state;
a path with a lowest initial cost of the transition costs to traverse from the starting state to a first of the one or more intermediate states; or
a path with a lowest individual transition costs to traverse from the starting state to the one or more intermediate states to the goal state.

6. The method of claim 5, wherein the path traversal strategy is selected for the human patient based on one or more preference associated with the human patient or resilience associated with the human patient.

7. The method of claim 1, wherein the pain experience states are associated with defined attributes based on one or more of: medication management, pain level, emotional state, or mobility; and
wherein the goal state is associated with an attribute that provides an improvement of one or more of: a sleep state, a mobility state, a medication state, an emotional state, or a pain level measurement.

8. The method of claim 1, wherein selecting the programming parameters for use in the neurostimulation device of the human patient includes a selection or recommendation of one or more program that includes the programming parameters; and
wherein each of the respective states used in the possible pathways is associated with a separate program used for the neurostimulation device that includes respective combinations of the programming parameters.

9. The method of claim 1, further comprising outputting the programming parameters to the neurostimulation device of the human patient, and wherein deployment of the programming parameters within one or more neurostimulation program causes a change in operation of the implantable electrical neurostimulation device.

10. The method of claim 1, wherein the implantable electrical neurostimulation device is further configured to treat the chronic pain condition by delivering at least one of: an electrical spinal cord stimulation, an electrical brain stimulation, or an electrical peripheral nerve stimulation, in the human patient; and
wherein the programming parameters cause a change in programming for the implantable electrical neurostimulation device for one or more of: pulse patterns, pulse shapes, a spatial location of pulses, waveform shapes, or a spatial location of waveform shapes, for modulated energy provided with a plurality of leads of the implantable electrical neurostimulation device.

11. A device to determine programming of an implantable electrical neurostimulation device for treating a chronic pain condition of a human patient, the device comprising:
at least one processor and at least one memory;
pain experience state evaluation circuitry, operable with the at least one processor and the at least one memory, configured to:
determine possible pathways to traverse pain experience states of the chronic pain condition, wherein the possible pathways provide respective paths among the pain experience states from a starting state to one or more intermediate states to a goal state;
determine transition costs between the pain experience states involved in each of the possible pathways, wherein respective states of the pain experience states are associated with different pain management characteristics based on therapy with the neurostimulation device;
identify a path of the possible pathways to reach the goal state, based on the transition costs and characteristics of the human patient; and
neurostimulation programming circuitry, in operation with the at least one processor and the at least one memory, configured to:
select programming parameters for use in the neurostimulation device of the human patient to cause a neurostimulation therapy, based on the identified path to achieve the goal state.

12. The device of claim 11, wherein the pain experience states are determined based on pain experience data collected from a population of patients, and wherein the transition costs are determined based on a frequency of transitions associated with the population of patients.

13. The device of claim 11, wherein to determine the possible pathways includes to identify the pain experience states and relative rankings of the pain experience states, and wherein the relative rankings are customized to the human patient.

14. The device of claim 13, wherein the relative rankings are customized to the human patient based on a measurement of: one or more preference associated with the human patient, or a resilience of the human patient.

15. The device of claim 11, wherein to identify the path to reach the goal state for the human patient includes use of a path traversal strategy provided from one of:
a shortest path independent of the transition costs from the starting state to the goal state;
a path with a lowest total cost of the transition costs to traverse from the starting state to the goal state;
a path with a lowest initial cost of the transition costs to traverse from the starting state to a first of the one or more intermediate states; or
a path with a lowest individual transition costs to traverse from the starting state to the one or more intermediate states to the goal state.

16. The device of claim 15, wherein the path traversal strategy is selected for the human patient based on one or more preference associated with the human patient or resilience associated with the human patient.

17. The device of claim 11, wherein the pain experience states are associated with defined attributes based on one or more of: medication management, pain level, emotional state, or mobility; and
wherein the goal state is associated with an attribute that provides an improvement of one or more of: a sleep state, a mobility state, a medication state, an emotional state, or a pain level measurement.

18. The device of claim 11, wherein to select the programming parameters for use in the neurostimulation device of the human patient includes a selection or recommendation of one or more program that includes the programming parameters; and
wherein each of the respective states used in the possible pathways is associated with a separate program used for the neurostimulation device that includes respective combinations of the programming parameters.

19. The device of claim 11, wherein the neurostimulation programming circuitry is further configured to output the programming parameters to the neurostimulation device of the human patient, and wherein deployment of the programming parameters within one or more neurostimulation program causes a change in operation of the implantable electrical neurostimulation device.

20. The device of claim 11, wherein the implantable electrical neurostimulation device is further configured to treat the chronic pain condition by delivering at least one of: an electrical spinal cord stimulation, an electrical brain stimulation, or an electrical peripheral nerve stimulation, in the human patient; and
wherein the programming parameters cause a change in programming for the implantable electrical neurostimulation device for one or more of: pulse patterns, pulse shapes, a spatial location of pulses, waveform shapes, or a spatial location of waveform shapes, for modulated energy provided with a plurality of leads of the implantable electrical neurostimulation device.
